(19) United States
(12) Patent Application Publication
(10) Pub. No.: US 2004/0010293 A1
(43) Pub. Date: Jan. 15, 2004

(54) Implantable cardiac stimulator and method for controling pacing dependent on the farfield ECG

(75) Inventors: Nils Holmstrom, Jarfalla (SE); Martin Obel, Danderyd (SE); Anders Bjorling, Jarfalla (SE); Sven-Erik Hedberg, Kungsangen (SE); Asa Uhrenius, Stockholm (SE); Goran Budgifars, Spanga (SE)

Correspondence Address: SCHIFF HARDIN & WAITE, 6600 SEARS TOWER, 233 S WACKER DR, CHICAGO, IL, 60606-6473, US

(73) Assignee: St. Jude Medical AB

(21) Appl. No.: 10/465,102

(22) Filed: Jun. 19, 2003

(30) Foreign Application Priority Data
Jul. 12, 2002 (SE) 0202214-3

(51) Int. Cl. A61N 1/362

(52) U.S. Cl. 607/9

(57) ABSTRACT

In a cardiac stimulating device and method for biventricular stimulation, the V-V interval between the stimulation pulses to the right and left ventricles is variable, and a farfield ECG obtained from measuring electrodes located outside of the heart is used for monitoring the mechanical synchronization between the right and left ventricles. The mechanical synchronization is optimized by an automatic adjustment of the V-V interval until the ORS duration is minimized.

### BACKGROUND OF THE INVENTION

1. Field of the Invention

The present invention relates to an implantable cardiac stimulating device and a pacing method. More precisely, the invention concerns such a stimulating device, and to a method operating such a device of the type having a housing, a control circuit enclosed in the housing, adapted to be connected to a first electrode to be positioned to stimulate a first ventricle of the heart. The control circuit is also adapted to be connected to a second electrode to be positioned to stimulate a second ventricle of the heart. The control circuit also delivers stimulating pulses. Furthermore, such a device has a sensor for sensing a farfield ECG similar to a surface ECG.

2. Description of the Prior Art

Most pacers are arranged to stimulate the right ventricle of the heart but it is also known to stimulate the left ventricle. In particular for treatment of congestive heart failure it is known to stimulate the left ventricle, or both ventricles, in order to optimize the hemodynamic performance of the heart. The improvement in hemodynamic performance that can be obtained is due to improved synchronization between the right and left ventricles.

U.S. Pat. No. 5,174,289 describes a method to sequentially pace the right ventricle at one or several locations to obtain the shortest possible QRS duration combined with an improved ventricular motion.

U.S. Pat. No. 6,233,082 describes a system and method for providing multiple chamber pacing of a patient's heart and in particular, pacing programmed for treatment of various forms of heart failure. Impedance sensing in the heart provides timing of mechanical contraction, and the pacemaker controls pacing to maintain bi-ventricular mechanical synchronization adjusted for maximum cardiac output.

U.S. Pat. No. 5,728,140 describes a method and an apparatus for pacing the left ventricle of the heart. The pacing electrode is positioned within the interventricular septum proximate to stimulate or sense the different chambers of the heart.

Also the article “A Method for Permanent Transvenous Left Ventricular Pacing” by Blanc et al, PACE Vol. 21, 1998, pp. 2021-2024, describes a method for positioning leads for left ventricular pacing.

The article “Four Chamber Pacing in Dilated Cardiomyopaty” by S Cazeau et al, PACE Vol. 17, 1994, pp 1974-1979, describes favorable clinical results with four chamber pacing. In one example QRS duration was found to decrease from 200 to 160 ms with four chamber pacing.

U.S. Pat. No. 4,928,688 describes a method and an apparatus for treating patients suffering from congestive heart failure by stimulating both the ventricles. The document discusses the problem involved when the left and right ventricles contract in asynchrony. In order to effect substantially simultaneous contraction of both ventricles, the document suggests means for separately processing sensed cardiac signals from each of the left and right ventricles. If ventricular contractions are not sensed in both ventricles within a period of coincidence defined by a time delay, the pacing pulse will be emitted at the end of this delay, but only to the ventricle for which a QRS-complex has not been sensed. The time delay is suggested to be in the order of 5-10 ms.

International publication PCT application WO 01/10499 A1 describes a biventricular pacer having means for measuring the evoked response signal from the right and left ventricle respectively. The time interval dT between the stimulation pulses to the right and left ventricles is adapted so that the evoked response signal from the two ventricles occur simultaneously.

### SUMMARY OF THE INVENTION

The purpose of pacing the left and right ventricles with separate leads is to improve the synchronization of the mechanical contraction of the two ventricles. The synchronization of the ventricles may be important for patients with severe congestive heart failure. These patients are often waiting for a heart transplant and optimal hemodynamic conditions during the time before the transplant is important for the outcome. A problem in this context is that synchronous pacing will not necessarily provide the best possible synchronization of the actual contraction of the ventricles. Impedance measurements have previously been used to determine mechanical synchronization of the ventricles. The present invention is based on the discovery that the use of impedance measurements is related to some problems in interpreting the impedance signal to determine optimal mechanical synchronization. The invention is based on the fact that optimal mechanical synchronization is present when the two ventricles are stimulated in a sequence that produces a surface ECG QRS with the shortest duration. Surface ECG QRS duration has been used to determine effectiveness of biventricular pacing but it has never been used for automatic adjustment of the time interval between pacing pulses to the two ventricles. The effect on QRS duration by biventricular pacing is described in the above referenced article by S Cazeau et al. It is thus an object of the present invention to provide an implantable cardiac stimulating device by means of which the synchronization of the ventricles is improved.

This object is achieved according to the invention in an implantable cardiac stimulating device wherein the stimulating pulses to the ventricles are delivered at such times that a farfield ECG measured at measuring points outside of the heart but inside the body gives the narrowest achievable QRS on a farfield ECG.

In a further embodiment of the invention the stimulating pulses to the ventricles are delivered at such times that the morphology of a farfield ECG signal measured at measuring points outside the heart fulfills certain predetermined morphological criteria.

In a further embodiment of the invention the implantable medical device has implantable electrodes located at a small distance from the implantation site but outside of the heart. The farfield ECG signal obtained from these electrodes is used for determination of the optimal interval dT between the stimulation pulses to the right and left ventricles respectively. These electrodes may be implemented as ring electrodes located on the electrode leads to be placed inside the heart.

### DESCRIPTION OF THE DRAWINGS

FIG. 1 shows schematically an implanted biventricular pacemaker system.

FIG. 2 shows schematically an implanted biventricular pacemaker system with far-field ECG monitoring through a short farfield ECG monitoring electrode.

FIG. 3 shows schematically an implanted biventricular pacemaker system with farfield ECG monitoring through an electrode dot located on the implanted pulse generator.

FIG. 4 shows schematically an implanted biventricular pacemaker system with far-field ECG monitoring through ring electrodes placed on the endocardial pacing electrodes.

FIG. 5 shows a schematic drawing of the control circuit in the implantable medical device.

FIG. 6 shows examples of surface ECG's for right ventricular pacing, left ventricular pacing and biventricular pacing respectively.

FIG. 7 shows an example of a ECG obtained with right atrial stimulation and stimulation of both ventricles.

### DESCRIPTION OF THE PREFERRED EMBODIMENTS

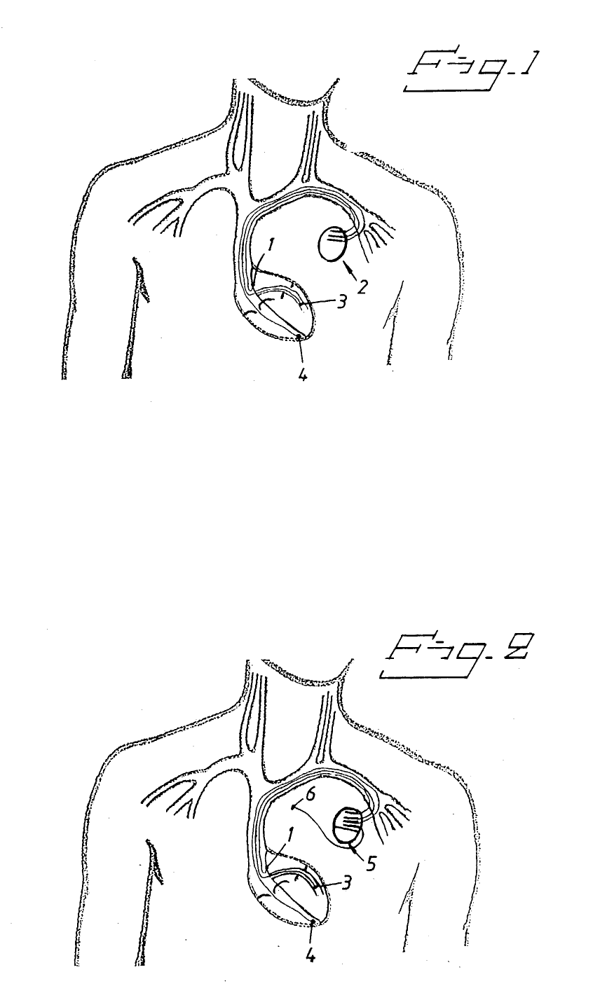

FIG. 1 shows a biventricular pacemaker as implanted. In this example an atrial electrode 1 is provided for atrial sensing/pacing by the implanted pulse generator 2. Further a left ventricular electrode 3 is provided for left ventricular sensing/pacing. The heart lead electrode 3 is implanted via the coronary sinus vein to a preferred position epicardially on the left ventricle. For right ventricular sensing/pacing a conventional right ventricular electrode 4 is provided. In some embodiments a fourth left atrial heart lead electrode is provided for left atrial sensing/pacing.

FIG. 2 shows a biventricular pacemaker according to the invention as implanted. A short non-endocardial electrode 6 is provided for sensing a far-field ECG from a position outside of the heart. In FIG. 2 it is assumed that the far-field ECG is sensed between the pulse generator 5 encapsulation and the short non-endocardial electrode 6. There may be several short non-endocardial electrodes to provide different farfield ECG sensing configurations. In the absence of atrial signals pacing pulses are delivered to the atrial electrode 1. Following an atrial sensed/paced event an AV interval is initiated. At the end of the AV interval pacing pulses are delivered to the right ventricular electrode 4 and to the left ventricular electrode 3. Following the delivery of the ventricular pacing pulses a far-field QRS is sensed via the short non-endocardial electrode 6 and the pulse generator 5 encapsulation. In an iterative search process the interval dT between the right ventricular pacing pulse and the left ventricular pacing pulse (which interval may be positive or negative depending on which pulse is delivered first) is varied until the evoked far-field QRS with the shortest duration is found. Alternatively the interval between the right ventricular pacing pulse and the left ventricular pacing pulse is varied until the evoked QRS-T fulfils predetermined morphological criteria such as the presence of a visible S-T segment. Other morphological criteria may be the surface under the positive and negative deflection of the QRS or the order in which negative and positive deflections occur or the duration of the most significant deflection in the far-field ECG signal. A far-field ECG configuration similar to configuration V1 in a surface ECG is an advantageous configuration even though any configuration can be used with appropriate adaptation of the analysis of the farfield ECG signal.

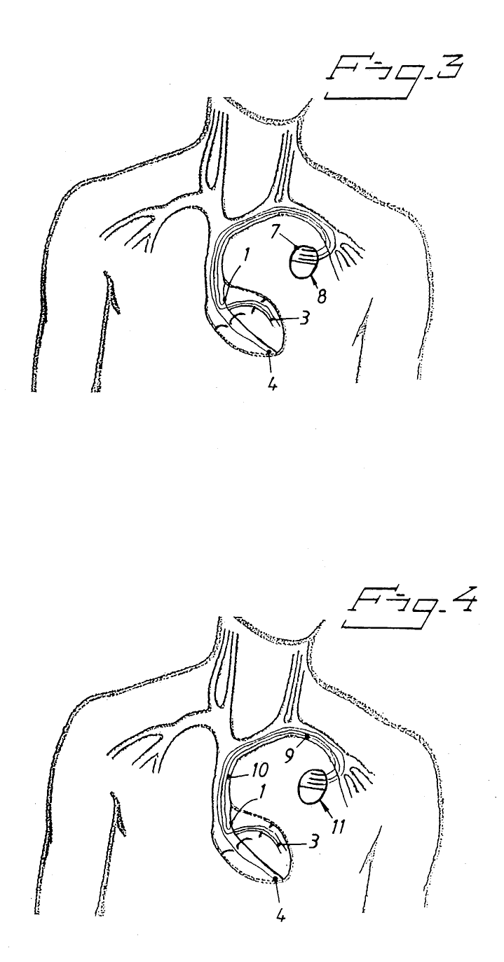

FIG. 3 shows a biventricular pacemaker according to the invention. A farfield ECG sensing electrode dot 7 is placed on the connector top and the farfield ECG is sensed between the dot 7 and the encapsulation of the pulse generator 8. In order to obtain several configurations of the farfield ECG several dots can be placed on the encapsulation and on the connector top. The functioning of the pulse generator descried in connection with FIG. 2 is applicable as well for FIG. 3.

FIG. 4 shows a biventricular pacemaker according to the invention. One or more of the endocardial heart electrode leads 1, 3 is provided with ring electrodes 9, 10 to be placed outside the heart for sensing a farfield ECG. A particularly advantageous method to connect the ring electrodes 9, 10 to the control unit of the pulse generator may be to use the future electrode connector standard IS-4 which provides 4 individual contacts for each IS-4 connector. The farfield surface ECG can be obtained between the pulse generator encapsulation and any of the ring electrodes 9,10 or between the ring electrodes 9, 10. This will provide several configurations for farfield ECG sensing.

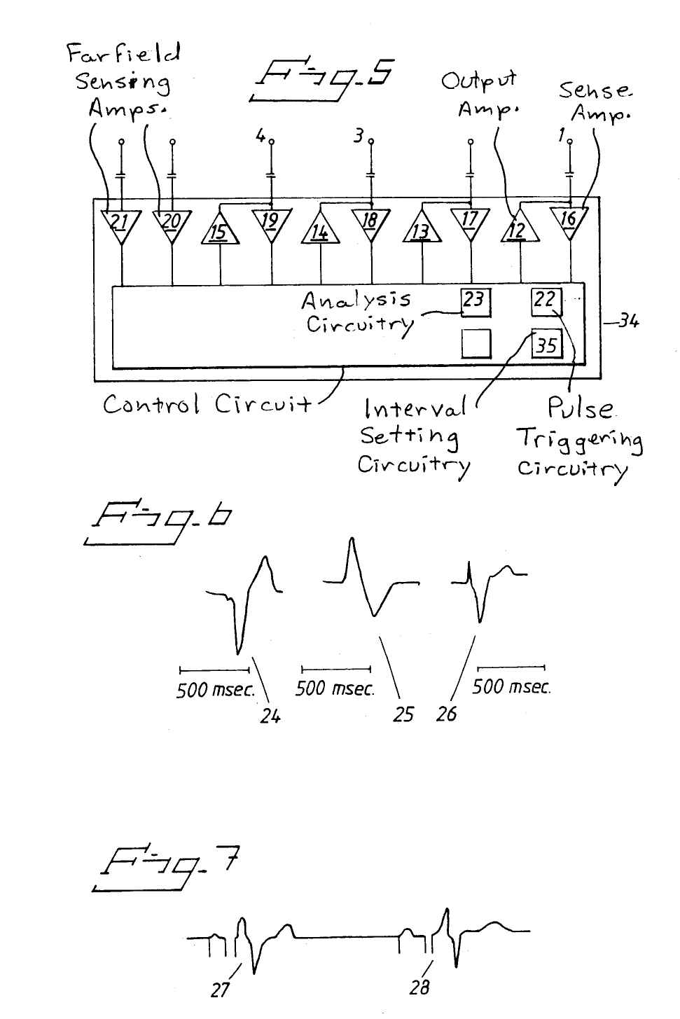

FIG. 5 shows a block diagram of a control circuit 34 used in the pulse generators shown in FIG. 4. The control circuit 34 has amplifiers 12, 13, 14, 15 for delivering stimulation pulses to electrodes 1, 3, 4. The control circuit 14 is adapted for a general configuration wherein the right and left atria as well as the right and left ventricles are paced. In many cases, however, one atrial electrode and two ventricular electrodes are present and the control circuit 34 also is usable in such an arrangement. The control circuit 34 further includes sense amplifiers 16, 17, 18, 19 for sensing atrial and ventricular activity respectively. Amplifiers 20 and 21 are used for sensing a farfield ECG signal from locations outside of the heart. The control circuit 34 also includes pulse triggering circuitry 22 which triggers the delivery of stimulating pulses to the first ventricular electrode 3 and the second ventricular electrode 4 within the same cycle of the heart with a time interval dT between the stimulating pulses. The time interval dT may be varied. Furthermore the control circuit 34 has analysis circuitry 23 for analyzing the farfield ECG signal obtained via the amplifiers 20 and 21. The analysis circuitry 23 may analyze the ORS duration, the QRS morphology, the duration of the most significant deflection, or ST segment visibility. In operation when biventricular pacing is executed the evoked ORS is analyzed for each biventricular stimulation. Further the control circuit 34 has interval setting circuitry means 35 for varying the interval dT in response to the farfield ECG analysis performed by the circuitry 23. In a search process the interval dT is varied and when the evoked QRS fulfills a certain criterion, the synchronization between the ventricles is considered to be optimal. The criterion may be minimum QRS duration or other morphological criteria.

FIG. 6 shows samples of a surface ECG (V1) obtained during pacing. Complex 24 is obtained during right ventricular pacing, complex 25 is obtained during left ventricular pacing, while complex 26 is obtained during biventricular simultaneous right and left ventricular stimulation. As can be seen in surface ECG configuration V1 the order of deflections are opposite when comparing LV and RV pacing. Other ECG configurations can also be employed for analyzing evoked QRS parameters that indicate synchronization between the ventricles. When biventricular pacing is applied the ORS is significantly shortened, the QRS has a unique morphology, there is a visible S-T segment. A farfield ECG obtained from a location under the skin is similar to a surface ECG obtained from a similar location on the surface of the skin. The surface ECG shows a greater variability due to polarization and contact problems with the ECG electrodes. This problem is not present with electrodes located under the skin for farfield sensing of the ECG signal. Thus a farfield ECG signal exhibits a better stability compared to a normal surface ECG signal.

FIG. 7 shows a schematic ECG indicating the function of a biventricular pacemaker utilizing the invention. In the first complex 27 an atrial stimulation pulse is indicated followed by biventricular stimulation pulses that are followed by the evoked QRS. The evoked QRS is obtained from surface ECG lead V1 but a similar signal can be obtained through electrodes implanted under the skin at a location close to the corresponding surface ECG lead. The evoked QRS is analyzed by the circuitry 23 in the control circuit 34. As a result of the analysis of the evoked QRS the interval dT between the ventricular pacing pulses is shortened in the next complex 28. The shortening results in a shorter evoked QRS and a longer S-T segment that indicates a better synchronization between the right and left ventricles. In this manner the pacemaker can make a search to find the optimal interval dT between the ventricular stimulation pulses. The sequence between the stimulation pulses depends on the electrode location and on the patient's myocardial condition. Thus in some patients the right ventricular stimulation pulse is followed by the left ventricular stimulation pulse and in some patients the sequence is reversed. The described search process, however, will find the optimal sequence and interval dT.

Although modifications and changes may be suggested by those skilled in the art, it is the intention of the inventors to embody within the patent warranted hereon all changes and modifications as reasonably and properly come within the scope of their contribution to the art.

We claim as our invention:

1. An implantable cardiac stimulating device comprising:
a housing;
a control circuit disposed in said housing;
a first electrode connected to said control circuit and adapted for positioning to interact with a first ventricle of a heart;
a second electrode connected to said control circuit and adapted for positioning to interact with a second ventricle of the heart;
a third electrode connected to said control circuit adopted for positioning at a distance from the heart for sensing a farfield ECG associated with the heart;
said control circuit supplying stimulation pulses respectively to said first and second ventricles via said first and second electrodes with a variable time interval, in a same cycle of the heart, between respective stimulations of the first and second ventricles; and
said control circuit analyzing a parameter associated with said farfield ECG as a measure of synchronization between said first and second ventricles and varying said time interval, dependent on said parameter, to optimize synchronization of said first and second ventricles.

2. An implantable cardiac stimulating device as claimed in claim 1 wherein said control circuit analyzes a QRS duration of said farfield ECG as said parameter.

3. An implantable cardiac stimulating device as claimed in claim 1 wherein said control circuit analyzes a QRS morphology of said farfield ECG as said parameter.

4. An implantable cardiac stimulating device as claimed in claim 1 comprising an extracardial lead on which said third electrode is disposed.

5. An implantable cardiac stimulating device as claimed in claim 1 wherein said housing has a conductive surface and wherein said third electrode comprises an electrode dot disposed on an exterior of said housing, insulated from said conductive surface, and senses said farfield ECG using said conductive surface as a counterelectrode.

6. An implantable cardiac stimulating device as claimed in claim 1 comprising an endocardial lead on which said third electrode is disposed as a ring electrode.

7. An implantable cardiac stimulating device as claimed in claim 6 wherein said endocardial lead carries one of said first and second electrodes.

8. A method for stimulating a heart comprising the steps of:
positioning a first electrode to interact with a first ventricle of a heart and connecting said first electrode to a control circuit;
positioning a second electrode to interact with a second ventricle of a heart and connecting said second electrode to said control circuit;
positioning a third electrode at a distance from the heart for sensing a far-field ECG associated with the heart and connecting said third electrode to said control circuit;
from said control circuit supplying stimulation pulses respectively to said first and second ventricles via said first and second electrodes with a variable time interval, in a same cycle of the heart, between respective stimulations of the first and second ventricles; and
analyzing in said control circuit a parameter associated with said farfield ECG as a measure of synchronization between said first and second ventricles and varying said time interval, dependent on said parameter, to optimize synchronization of said first and second ventricles.

9. A method as claimed in claim 8 comprising analyzing a QRS duration of said farfield ECG as said parameter.

10. A method as claimed in claim 8 comprising analyzing a QRS morphology of said farfield ECG as said parameter.

11. A method as claimed in claim 8 comprising disposing said third electrode on an extracardial lead.

12. A method as claimed in claim 8 comprising:
containing said control circuit in an implantable housing having a conductive surface;
forming said third electrode as an electrode dot disposed on an exterior of said housing insulated from said conductive surface; and
sensing said farfield ECG between said electrode dot and said conductive surface.

13. A method as claimed in claim 8 comprising employing a ring electrode disposing said third electrode on an endocardial lead as a ring electrode.

14. A method as claimed in claim 13 comprising carrying one of said first and second electrodes on said endocardial lead.
